(19) United States
(12) Patent Application Publication
(10) Pub. No.: US 2026/0224904 A1
(43) Pub. Date: Aug. 6, 2026

(54) LEAD BODY WITH THERMOPLASTIC POLYURETHANE ADHESIVE

(71) Applicant: Heraeus Medical Components LLC, Fridley, MN (US)

(72) Inventor: Richard KONISZCZUK, Fridley, MN (US)

(73) Assignee: Heraeus Medical Components LLC, Fridley, MN (US)

(21) Appl. No.: 19/037,028

(22) Filed: Jan. 24, 2025

(51) Int. Cl. A61N 1/375; B29C 45/14; H01R 24/58

(52) U.S. Cl. CPC A61N 1/3752; B29C 45/14065; B29C 45/14549; B29C 45/14639; H01R 24/58; B29C 2045/14131; H01R 2201/12

(57) ABSTRACT

One aspect is a method of forming a lead body comprising providing a plurality of individual conductors between a proximal end and a distal end of the lead body. Each one of the plurality of individual conductors is coupled to one electrode of a plurality of electrodes. An outer cover is placed over the plurality of individual conductors and over the electrodes in a substantially cylindrical shape with an outer diameter, leaving a distal opening in the cover at the distal end, the distal opening having a same diameter as the outer cover. The outer cover comprises a first thermoplastic material that hermetically seals the lead body. An adhesive cap is applied to the outer cover and fully over the distal opening thereby closing the distal end, wherein the adhesive comprises the first thermoplastic material.

### BACKGROUND

The discussion below is merely provided for general background information and is not intended to be used as an aid in determining the scope of the claimed subject matter. Many leads, and especially multipolar leads for medical devices, are usually very complex to manufacture, because their preparation requires many different components that must be assembled in many steps. Manufacturing is even more complex for small-sized leads, which are particularly desired in the field of medical devices. The complex design of multipolar leads and the challenging manufacturing processes for preparing these leads typically results in high prices for the end product.

Some complex multipolar leads are first assembled coupling multiple conductors to multiple electrodes, and then an outer coating or jacket is slid over or extruded over the assembled multipolar leads. It is important that the outer coating or jacket is fully sealed over the multiple conductors and electrodes, including at the ends, which are typically open in order to complete the assembly process. Although various techniques are used to close the openings of the outer coating or jacket, there are shortcomings.

For example, where the outer coating is a thermoplastic material, it can be melted and re-flowed to close off openings. However, this re-flow process can degrade the outer dimensions of the lead. Furthermore, the process can crowd or deform the conductors within the lead, which can cause shorts or faults. Such lower reliability can lead to a higher risk of failure for the lead, which in turn can have significant consequences, e.g. for the health of a patient when the lead is used in a medical device. For these and other reasons, there is a need for the present embodiments.

### SUMMARY

This Summary and the Abstract below are provided to introduce a selection of concepts in a simplified form that are further described below in the Detailed Description. This Summary and the Abstract are not intended to identify key features or essential features of the claimed subject matter, nor are they intended to be used as an aid in determining the scope of the claimed subject matter. The claimed subject matter is not limited to implementations that solve any or all disadvantages noted in the Background.

One aspect is a method of forming a lead body comprising providing a plurality of individual conductors between a proximal end and a distal end of the lead body. Each one of the plurality of individual conductors is coupled to one electrode of a plurality of electrodes. An outer cover is placed over the plurality of individual conductors and over the electrodes in a substantially cylindrical shape with an outer diameter, leaving a distal opening in the cover at the distal end, the distal opening having a same diameter as the outer cover. The outer cover comprises a first thermoplastic material that hermetically seals the lead body. An adhesive cap is applied to the outer cover and fully over the distal opening thereby closing the distal end, wherein the adhesive comprises the first thermoplastic material.

One embodiment is a method of forming a lead body comprising:

providing a plurality of individual conductors between a proximal end and a distal end of the lead body;

coupling each one of the plurality of individual conductors to one electrode of a plurality of electrodes;

placing an outer cover over the plurality of individual conductors and over the plurality of electrodes in a substantially cylindrical shape with an outer diameter, leaving a distal opening in the cover at the distal end, the distal opening having a same diameter as the outer cover;

wherein the outer cover comprises a first thermoplastic material that hermetically seals the lead body; and

applying an adhesive cap to the outer cover and fully over the distal opening thereby hermetically closing the distal end, wherein the adhesive comprises the first thermoplastic material.

Another embodiment is a method of a previous embodiment, wherein placing the outer cover over the plurality of individual conductors and over the electrodes further leaves a proximal opening in the outer cover at the proximal end, the proximal opening having a same diameter as the outer cover.

Another embodiment is a method of a previous embodiment, further comprising applying an adhesive cap to the outer cover and fully over the proximal opening thereby closing the proximal end, wherein the adhesive comprises the first thermoplastic material.

Another embodiment is a method of a previous embodiment, wherein applying the adhesive cap to the outer cover and over the proximal and distal openings does not change the diameter of either the proximal or distal opening.

Another embodiment is a method of a previous embodiment, further comprising coupling each one of the plurality of individual conductors to one proximal ring of a plurality of proximal rings.

Another embodiment is a method of a previous embodiment, wherein the first thermoplastic material is a medical grade thermoplastic polyurethane elastomer.

Another embodiment is a method of a previous embodiment, wherein the outer cover is not melted or reflowed at any point in the forming of the lead body or wherein the lead and the outer cover are never subjected to a temperature over 330°F in the forming of the lead body.

Another embodiment is a method of a previous embodiment, wherein placing the outer cover over the plurality of individual conductors comprises extruding the outer cover.

Another embodiment is a method of a previous embodiment, wherein applying an adhesive caps includes dipping both the distal and proximal ends of the lead into liquid thermoplastic polyurethane elastomer adhesive.

Another embodiment is a method of a previous embodiment, wherein applying an adhesive caps includes initially dipping the distal or proximal ends of the lead into liquid thermoplastic polyurethane elastomer adhesive having a first percent solids content of polyurethane and then subsequently dipping the distal or proximal ends of the lead into liquid thermoplastic polyurethane elastomer adhesive having a second percent solids content of polyurethane, wherein the second solids content percent is larger than the first.

Another embodiment is a method of a previous embodiment, wherein applying adhesive caps includes initially dipping the distal or proximal ends of the lead into liquid thermoplastic polyurethane elastomer adhesive and then subsequently applying a mold release agent and placing the distal end into a mold cavity to shape the end.

Another embodiment is a method of a previous embodiment, wherein applying adhesive caps includes initially dipping the distal or proximal ends of the lead into liquid thermoplastic polyurethane elastomer adhesive, the adhesive comprising a polyurethane solid dissolved in a solvent, the solids content having a percent range from 2 percent to 30 percent.

Another embodiment is a method of a previous embodiment, wherein the liquid thermoplastic polyurethane elastomer adhesive includes a solvent such that the adhesive penetrates up into the plurality of conductors under capillary action.

One embodiment is a lead body comprising:

a plurality of individual conductors between a proximal end and a distal end of the lead body;

a plurality of electrodes, wherein each one electrode of the plurality of electrodes is coupled to each one of the plurality of individual conductors;

an outer cover over the plurality of individual conductors and over the electrodes in a substantially cylindrical shape with an outer diameter, the outer cover having a distal opening in the cover at the distal end, the distal opening having a same diameter as the outer cover;

wherein the outer cover comprises a first thermoplastic material that hermetically seals the lead body; and

an adhesive cap coupled to the outer cover and fully over the distal opening thereby closing the distal end, wherein the adhesive comprises the first thermoplastic material.

Another embodiment is a lead body of a previous embodiment, wherein the outer cover further comprises a proximal opening in the outer cover at the proximal end, the proximal opening having a same diameter as the outer cover.

Another embodiment is a lead body of a previous embodiment, further comprising an adhesive cap coupled to the outer cover and fully over the proximal opening thereby closing the proximal end, wherein the adhesive comprises the first thermoplastic material.

Another embodiment is a lead body of a previous embodiment, wherein the adhesive cap coupled the outer cover and over the proximal and distal openings has the same diameter as the diameter of both the proximal and distal openings.

Another embodiment is a lead body of a previous embodiment, further comprising a plurality of proximal rings, wherein each ring of the plurality of proximal rings is coupled to one of the plurality of individual conductors.

Another embodiment is a lead body of a previous embodiment, wherein the first thermoplastic material is a medical grade thermoplastic polyurethane elastomer.

### BRIEF DESCRIPTION OF THE DRAWINGS

FIG. 1 illustrates a partially ghosted lead body in accordance with one embodiment.

FIG. 2 is a sectional view of the lead body of FIG. 1 in accordance with one embodiment.

FIG. 3 illustrates a partially formed lead body with an outer cover in accordance with one embodiment.

FIG. 4 illustrates a partially formed lead body with an adhesive cap in accordance with one embodiment.

FIG. 5 illustrates a lead body in accordance with one embodiment.

FIG. 6 illustrates a mold with a concave cavity for forming a radiused end according to one embodiment.

FIG. 7A illustrates a distal end of a lead body in accordance with the prior art.

FIG. 7B illustrates a distal end of a lead body in accordance with one embodiment.

FIG. 8 illustrates a close-up end view of an adhesive cap at a proximal end of a lead body in accordance with one embodiment.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise. It is to be understood that other embodiments may be utilized and structural or logical changes may be made without departing from the scope of the present embodiments. The following detailed description, therefore, is not to be taken in a limiting sense, and the scope of the present embodiments are defined by the appended claims.

It is to be understood that the features of the various exemplary embodiments described herein may be combined with each other, unless specifically noted otherwise.

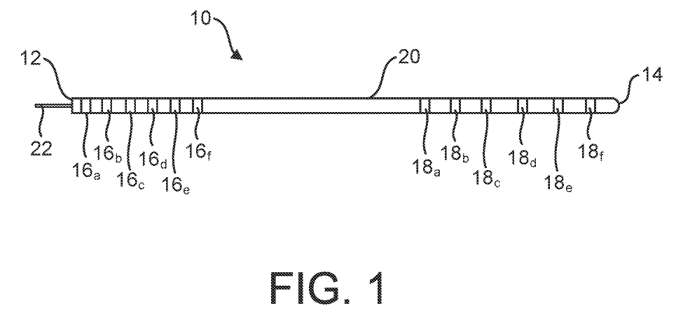

FIG. 1 is a lead body 10 in accordance with one embodiment. In one embodiment, lead body 10 includes proximal end 12, distal end 14, a plurality of proximal rings 16a-f, a plurality of distal rings 18a-f and an outer cover 20. In one embodiment, lead body also includes a guidewire 22 extending through its center lumen 24.

In one embodiment, outer cover 20 may be a tube-shaped sleeve. Proximal and distal rings 16a-f/18a-f may be a ring shaped or annular. Rings 16a-f/18a-f may fully surround the lead body 10 or extend only along a part of the lead body, in one embodiment along a major part of the lead body 10. Although six proximal rings 16a-f and distal rings 18a-f are illustrated in FIG. 1, in various embodiments, lead body 10 may include 2, 4, 6, 8, 10, 12 or more proximal rings 16a-f and distal rings 18a-f.

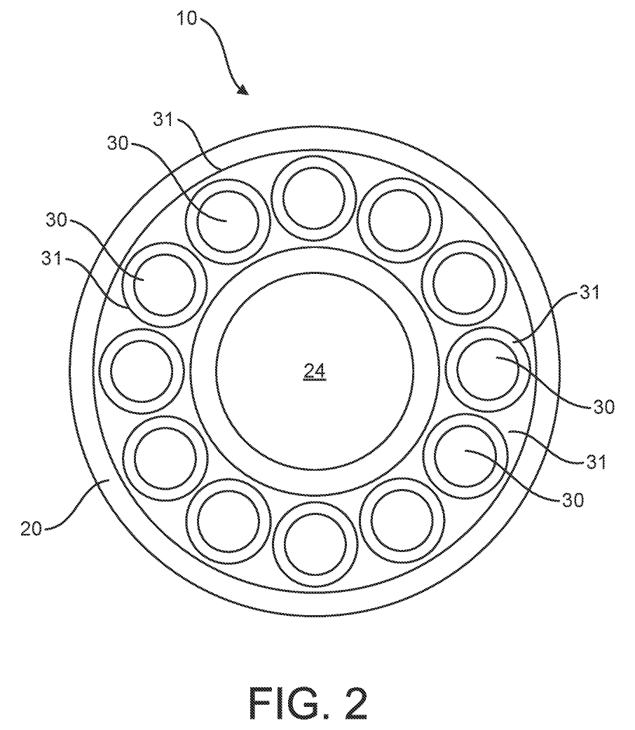

FIG. 2 is a sectional view of a lead body 10 in accordance with one embodiment. In one embodiment, lead body 10 includes a plurality of conductors 30, which extend between the proximal end 12 and distal end 14 and are wound about a center lumen 24. In one embodiment, each conductor of plurality of conductors 30 is a conductive wire that is individually isolated with a layer of insulation 31. In this way, each conductor 30 is electrically isolated from each other conductor 30. In the illustration of FIG. 2, twelve conductors 30 are illustrated, such one conductor is coupled between one of twelve proximal rings 16 and one of twelve distal rings 18. Again, in various embodiments, lead body 10 may include 2, 4, 6, 8, 10, 12 or more proximal rings 16 and distal rings 18, and a corresponding 2, 4, 6, 8, 10, 12 or more conductors connecting them.

In one embodiment, one conductor of the plurality of conductors 30 is coupled between one proximal ring 16 and one distal ring 18. For example, in the embodiment of FIG. 1, proximal ring 16a is coupled to distal ring 18a by a conductor 30, proximal ring 16b is coupled to distal ring 18b by a separate conductor 30, proximal ring 16c is coupled to distal ring 18c by a separate conductor 30, proximal ring 16d is coupled to distal ring 18d by a separate conductor 30, proximal ring 16e is coupled to distal ring 18e by a separate conductor 30, and proximal ring 16f is coupled to distal ring 18f by a separate conductor 30.

In this way, each proximal ring 16 is electrically coupled to one single distal ring 18 via an independent conductor 30. In FIGS. 2, 12 conductors 30 are illustrated, such that each conductor couples one of 12 proximal rings 16 to one of 12 distal rings 18. Because each conductor 30 is surrounded by insulation 31, each proximal ring 16 that is electrically coupled to each distal ring 18 remains electrically isolated from all other rings 16/18.

In one embodiment, lead body 10 is configured for use within the body of a mammal or human. In one embodiment, the plurality of distal rings 18 are electrodes that are configured for sensing and/or stimulation within a biological application. In some embodiments, the electrodes are provided toward distal end 14 of the lead body 10 for sensing and/or stimulation within a human body. Distal end 14 is placed adjacent tissue that is to be sensed or stimulated and the electrodes either transmit or receive energy. In one embodiment, the plurality of proximal rings 16 are respective connectors or contacts, which are electrically coupled to the electrodes, are provided on the proximal end 12 of a lead 10 for plugging in to a medical device.

A lead body 10 with ring electrodes 18 can be used as a medical device in electrophysiology and neurostimulation procedures to deliver electrical signals to specific areas of the body, typically within the heart, nervous system, or other tissue types. Lead body 10 is a long, thin, and flexible tube designed to be inserted into the body through a vein or artery, or directly into a targeted tissue (e.g., the heart or nervous system). Lead body 10 is designed to navigate through complex anatomical structures. It is typically advanced using imaging techniques like fluoroscopy or echocardiography, to ensure precise positioning.

Lead body 10 in accordance with embodiments described herein, allow for the manufacture of leads having increased density of electrode segments. Increased density of electrode segments is useful in a variety of applications. For example, lead 10 can be used in deep brain stimulation (DBS), in which lead 10 delivers electrical pulses into one or several specific sites within the brain of a patient to treat various neurological disorders, such as chronic pain, tremors, Parkinson’s disease, dystonia, epilepsy, depression, obsessive-compulsive disorder, and other disorders. In other applications, lead 10 may be configured for spinal cord stimulation, peripheral nerve stimulation, dorsal root stimulation, cortical stimulation, ablation therapies, cardiac rhythm management leads, various catheter configurations for sensing, and various other therapies where directional sensing or stimulation are needed.

Lead body 10 may be assembled in a variety of ways in accordance with various embodiments. In one embodiment, lead body 10 includes the plurality of conductors 30 that are helically wound about center lumen 24. Next, one conductor 30 is coupled to one proximal ring 16 and to one distal ring 18. Connection of each of the remaining conductors 30 is them completed between one proximal ring 16 and one distal ring 18, until each conductor 30 couples one proximal to one distal ring 16/18.

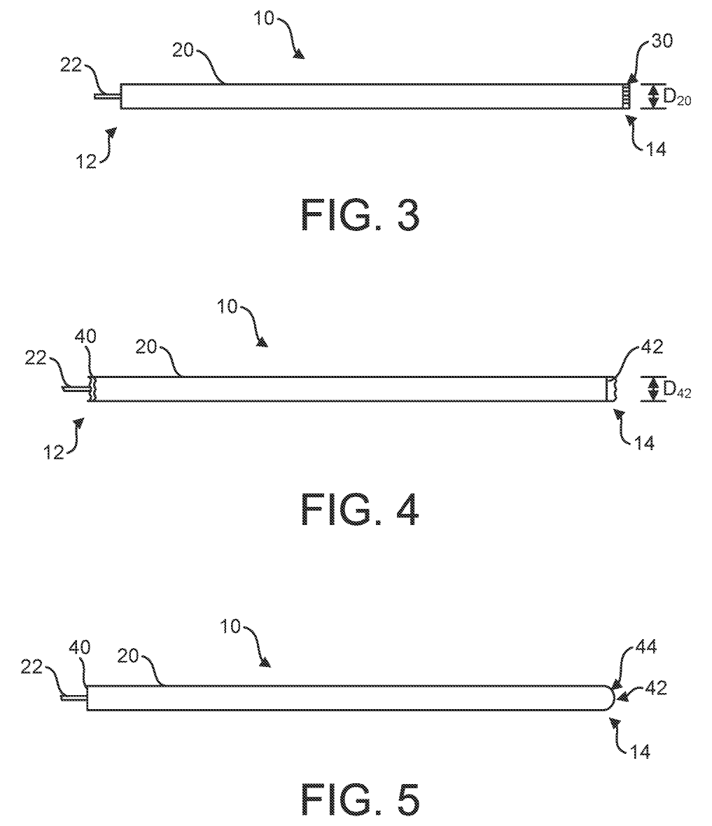

After all connections are made, outer cover 20 is added over the various components in order to seal lead body 10. FIG. 3 illustrates outer cover 20 added over the plurality of proximal rings 16, the plurality of distal rings 18, and the plurality of conductors 30. In one embedment, outer cover 20 is generally cylindrically shaped and is slid over the outer diameter of the plurality of conductors 30. In another embodiment, outer cover 30 is extruded over the outer diameter of the plurality of conductors 30. The design of lead 10 also allows for attaching the proximal rings 16 and distal rings 18 at any desired position along its length. Once the plurality of proximal rings 16, the plurality of distal rings 18, and the plurality of conductors 30 are all connected as desired in any customizable way, cover 20 is added over them.

In one embodiment, outer cover 20 is a medical grade thermoplastic polyurethane elastomer. A medical grade thermoplastic polyurethane elastomer can provide a good hermetic seal for lead body 10 and has other favorable characteristics that will be discussed further below. In various embodiments, several types of thermoplastic polyurethane elastomers can be used, such as 65D, 75D, 80A, 90A, and Pellethane®.

In one embodiment, outer cover 20 is assembled over the plurality of proximal rings 16, the plurality of distal rings 18, and the plurality of conductors 30 as a cylindrically-shaped tubular member, and such that outer cover 20 of lead body 10 remains fully open at both its proximal and distal ends 12/14. Cover 20 has a diameter D₂₀ that defines the diameter of the openings at both its proximal and distal ends 12/14. Because lead body 10 will be configured for use within a human or mammal body, these openings in the proximal and distal ends12/14 must be hermitically closed in order to complete lead body 10 for use in a medical application.

Because lead body 10 and its components, such as the plurality of proximal rings 16, the plurality of distal rings 18, and the plurality of conductors 30 are very small, it is important that these components within cover 20 are not compromised during the closing of the openings in cover 20 in the proximal and distal ends 12/14. For example, it is important that the ends are not compressed such that the plurality of conductors 30 are compressed together, which would increase the risk of an electrical short. For example, in one embodiment, the diameter of conductors 30, including insulation layer 31, is in a range from about 0.08mm/.003inch to about 0.3mm/0.012inch. In one embodiment, the overall diameter of lead body 10, including cover 20, is in a range from about 0.65mm/.025inches to 5mm/0.197inch. With these very small dimensions, it is important not to squeeze or compress the conductors.

FIG. 4 illustrates lead body 10, where the openings in outer cover 20 at the proximal and distal ends 12/14 have been closed in accordance with one embodiment. In one embodiment, the openings in cover 20 at the proximal and distal ends 12/14 are closed off by application of an adhesive caps 40/42, which covers the entire openings. As illustrated in FIG. 4, the diameter D₄₂ of both adhesive caps 40/42 (only the diameter of cap 42 is marked for simplicity of illustration, but it is the same as cap 40) are the same as the diameter D₂₀ of cover 20. Importantly, application of adhesive caps 40/42 to cover 20 does not alter in any way the diameter D₂₀ of cover 20. Cover 20 is not compressed with the application of adhesive caps 40/42.

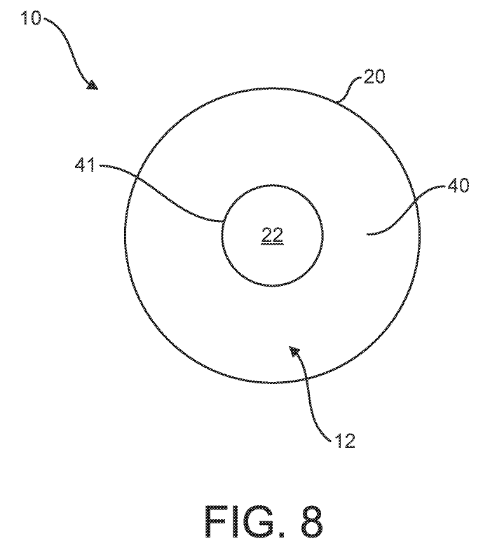

In one embodiment, the adhesive caps 40/42 are an adhesive made of medical grade thermoplastic polyurethane elastomers. In one embodiment, the proximal and distal ends 12/14 and dipped into a liquid form of the adhesive to form the adhesive caps 40/42, such that the adhesive caps 40/42 adhere to the outer cover 20 and completely seals the lead body 10 with a hermetic seal at both its proximal and distal ends 12/14. In one embodiment, proximal tip 12 is sealed with adhesive cap 40 that surrounds guidewire 22 thereby maintaining an open inner diameter for guidewire 22. In one embodiment, the guidewire 22 is coated with Teflon® or the like to prevent adhesion with the applied adhesive cap 40, such that the adhesive cap 40 seals off the proximal end opening, but still allows the guidewire 22 to move in and out of center lumen 24 (see, for example, FIG. 8).

In one embodiment, the distal tip 14 is completely sealed with adhesive cap 42. The adhesive material is cured, which creates a leak proof seal for both proximal and distal ends 12/14. In one embodiment, proximal tip 12 is sealed with adhesive cap 40 prior to distal tip 14 being closed off. In one alternative embodiment, no guidewire 22 is provided and both the proximal tip 12 and the distal tip 14 are each completely sealed respectively with adhesive caps 40/42. In such embodiment, adhesive cap 40 is the mirror image of adhesive cap 42, such as illustrated in FIGS. 1, 5, and 7B.

In one embodiment, the adhesive caps 40/42 are an adhesive made of medical grade thermoplastic polyurethane elastomers, in which the polyurethane is dissolved in a solvent. In one embodiment, in order to keep the adhesive from being too viscous, the amount of polyurethane solids dissolved in the solvents are limited. In one embodiment, the percent of polyurethane solids in the adhesive is in a range from 2% - 30%, with the remaining percentage being solvent.

In one embodiment, any of a variety of solvents that can dissolve polyurethanes can be used for the adhesive for adhesive caps 40/42. For example, the adhesive could use as a solvent Dimethyl sulfoxide (DMSO), Dimethylacetamide (DMAC), Dimethylformamide (DMF), Tetrahydrofuran (THF), or other similar solvent that will also dissolve polyurethanes.

In one embodiment, one or both of adhesive caps 40/42 are applied in a two-or-more step dipping process. In one embodiment, Lead body 10, such as illustrated in FIG. 3, had its proximal and distal ends 12/14 initially dipped in an adhesive having a relatively low solids content. For example, proximal and distal ends 12/14 of lead body 10 may initially be dipped in an adhesive having 2 percent polyurethane solids with 98 percent solvent. Because such a formulation will be relatively non-viscous, it will readily flow over and cover the opens at proximal and distal ends 12/14. After dipping, the applied adhesive is then allowed to flash dry.

Subsequently, proximal and distal ends 12/14 are then dipped in an adhesive have a relatively higher solids content, such as an adhesive having 4 percent polyurethane solids with 96 percent solvent. This subsequent adhesive will be slightly more viscous, and this added viscosity will help form a smooth and somewhat rounded shape for adhesive caps 40/42. In other embodiments, proximal and distal ends 12/14 may continue to be repeatedly dipped in an adhesive having a relatively increasing solids content with each subsequent dip.

FIG. 5 illustrates lead body 10 with adhesive cap 42 at distal end 14 further radiused to improve its shape in accordance with one embodiment. In one embodiment, cap 42 is smoothed into a convex-shaped distal end. Smoothing the adhesive cap 42 can be useful in ensuring that lead body 10 can be easily and cleanly inserted into a human vasculature without any unevenness that could otherwise cause obstacles in pushing in the lead 10 into and through the vasculature.

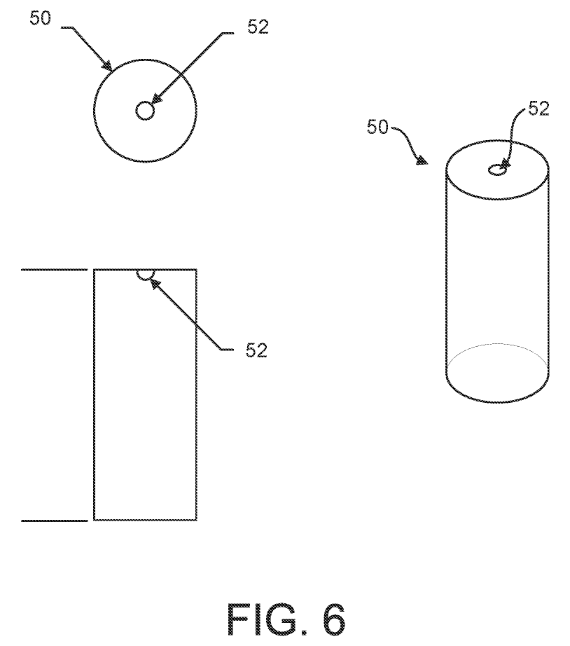

In one embodiment, after the adhesive cap 42 is applied, such as with the above-described dipping process, a relatively low temperature can be applied in order to facilitate smoothing cap 42. FIG. 6 illustrates a mold 50 having a concave cavity 52 into which cap 42 is placed in order to form the radiused end according to one embodiment. For example, temperatures of between 260°F to 300°F can be used with an approximately 0.5 second heat soak time, while adhesive cap 42 is placed into cavity 52.

Importantly, the temperature used for smoothing the adhesive cap 42 is held well below the melting point of the cover 20, such that the cover 20 is prevented from re-flowing. In one embodiment, where outer cover 20 is thermoplastic polyurethane elastomer, the temperature used to smooth the cap 42 is well below the 330°F needed to re-flow cover 20. In this way the opening in cover 20, over which adhesive cap 42 is placed, never changes in diameter due to the application of temperature in conjunction with smoothing cap 42. In one embodiment, both proximal and distal tips 12/14 are radiuses to improve application of the device.

In one embodiment, an additional step can be used during the tipping process. In one embodiment, distal end 14 is first dipped in an adhesive to create the cured rough ball tip. Next, a urethane mold release agent is then applied just before distal end 14 is placed into concave cavity 52 of mold 50. The application of a urethane mold release agent just prior to placement in to mold 50 aids in smoothing the radiused ball of adhesive cap 42, improving both its feel and appearance.

In various embodiments, a number of different release agents may be used to assist with the clarity and smoothness of adhesive cap 42. For example, mold release agents such as Hexane, Acetone, Nix Stix, Stoner, Slide, Quick, or other mold release agent in either aerosol or liquid form can be used.

In one embodiment, because the outer cover 20 and the adhesive caps 40/42 are both medical grade thermoplastic polyurethane, when the adhesive caps 40/42 are applied to the outer cover 20, there is an interlaced bond between the thermoplastic polyurethane materials of the cover 20 and of the adhesive caps 40/42. As such, there is excellent adhesion between them. Furthermore, because the adhesive caps 40/42 are applied to cover 20 in a liquid form, the diameter D₂₀ of the openings in outer cover 20 at the proximal and distal ends 12/14 is maintained and not compromised, compressed, or changed in any way. In one embodiment, the adhesive caps 40/42 are applied to cover 20 without the use of any applied heat. Accordingly, the diameter D₂₀ of the openings at the ends of outer cover 20 are also not altered by applied heat that would re-flow the cover 20.

In one embodiment, using medical grade thermoplastic polyurethane elastomer adhesive allows for lead body 10 to be sealed from ingress without the use of heat. The inventors found that use of thermoplastic polyurethane elastomer adhesive on a thermoplastic polyurethane elastomer cover offers zero shorts in high potential (Hi-Pot) testing. This is critically important for applications such as lead body 10 used as a neuromodulation lead. Testing shows this vastly improved over prior systems, and reduced failure rates caused by gaps created when trying to join reflow tubing for the outer cover.

Testing also showed that use of thermoplastic polyurethane elastomer adhesive on outer covers that were not of similar materials, such as Ethylene tetrafluoroethylene (ETFE), did not provide dependable or long-lasting bonds. Although many prior art leads use ETFE for over layer covers, thermoplastic polyurethane adhesive tends to peal away from ETFE after extended use. The unique use of a thermoplastic polyurethane elastomer adhesive on a thermoplastic polyurethane elastomer cover, however, provides excellent bonding and reliable hermetic seals for lead body 10, which is critical for medical applications in which lead body 10 is inserted into the human vasculature.

The inventors found that using an adhesive applied to cover 20 as a liquid with a solvent, including dipping the outer cover 20 into the adhesive, allows the adhesive to readily flow into any irregularities the surface of cover 20 may have. The adhesive is then flash cured sealing all possible vacancies as it chemically bonds to the thermoplastic materials, which is unlike any reflow process of an outer cover. The reflow process requires heat and compression to melt the materials of the outer cover in over to create the bond.

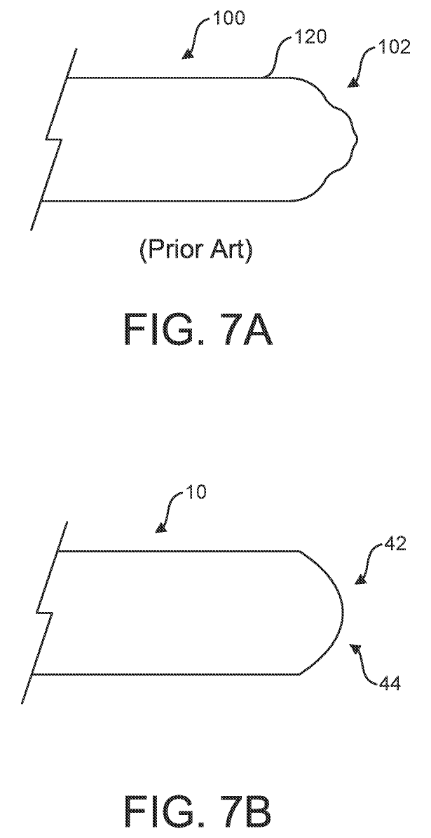

In prior systems, an outer cover to a lead may be closed by heating the cover until the it re-flows, and then covers the openings at its ends. FIG. 7A illustrates a close-up view of the result of such a re-flow process. A lead 100 has an opening of a cover 120 that is heating and re-flowed to close the opening at the tip 102. The re-flowed tip 102 compresses the tip 102 and also compresses the components within it, such as the conductors. This increases the risk of failure and shorting. The diameter of the opening in cover 120 will actually be reduced after the re-flow process, and accordingly, there is less space for the components within it, such as the conductors. This squeezing down on the components within the outer cover will increase the likelihood of shorts and faults.

FIG. 7B illustrates a comparative close-up view of adhesive cap 42 of one embodiment, which has been radiused in accordance with one embodiment. Because the outer cover 20 is never subjected to sufficient heat to re-flow or melt it, the diameter D₂₀ of the opening over which the cap 42 is placed remains unchanged. Accordingly, the ends of lead 10 are not compressed, such that the plurality of conductors 30 are never compressed together, thereby decreasing risk of failure.

FIG. 8 illustrates a close-up end view of adhesive cap 40 at proximal end 12 of lead body 10 in accordance with one embodiment. In one embodiment, application of cap 40 as a liquid adhesive over the opening at proximal end 12 of outer cover 20 has various advantages in sealing lead body 10. In one embodiment, when liquid adhesive is applied at the proximal end 12, not only does it close off the opening at proximal end 12 of outer cover 20, but it also draws into the lead 10. In one embodiment, when applied, the liquid adhesive with solvent, under capillary action, actually penetrates down (into the page as illustrated in FIG. 8) the conductors 30, which improves the seal for lead body 10. The solvent function creates this action and the polyurethane in liquid form allows movement of this fluid up the conductors 30 and surrounds the individual conductors 30 and establishes a bond, creating a superior bond.

In one embodiment, lead body includes an inner lumen into which a guidewire 22 or the like can be placed. Adhesive cap 40 provides a seal between the outer cover 20 and the outer diameter 41 of the inner lumen. Accordingly, lead body is fully sealed at its proximal end 12, which still allowing a guidewire 22 or the like to enter into the inner lumen without sacrificing an excellent hermetic seal.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A method of forming a lead body comprising:
providing a plurality of individual conductors between a proximal end and a distal end of the lead body;
coupling each one of the plurality of individual conductors to one electrode of a plurality of electrodes;
placing an outer cover over the plurality of individual conductors and over the plurality of electrodes in a substantially cylindrical shape with an outer diameter, leaving a distal opening in the cover at the distal end, the distal opening having a same diameter as the outer cover;
wherein the outer cover comprises a first thermoplastic material that hermetically seals the lead body; and
applying an adhesive cap to the outer cover and fully over the distal opening thereby hermetically closing the distal end, wherein the adhesive comprises the first thermoplastic material.

2. The method of claim 1, wherein placing the outer cover over the plurality of individual conductors and over the electrodes further leaves a proximal opening in the outer cover at the proximal end, the proximal opening having a same diameter as the outer cover.

3. The method of claim 2 further comprising applying an adhesive cap to the outer cover and fully over the proximal opening thereby closing the proximal end, wherein the adhesive comprises the first thermoplastic material.

4. The method of claim 3, wherein applying the adhesive cap to the outer cover and over the proximal and distal openings does not change the diameter of either the proximal or distal opening.

5. The method of claim 1 further comprising coupling each one of the plurality of individual conductors to one proximal ring of a plurality of proximal rings.

6. The method of claim 1, wherein the first thermoplastic material is a medical grade thermoplastic polyurethane elastomer.

7. The method of claim 1, wherein the outer cover is not melted or reflowed at any point in the forming of the lead body or wherein the lead and the outer cover are never subjected to a temperature over 330°F in the forming of the lead body.

8. The method of claim 1, wherein placing the outer cover over the plurality of individual conductors comprises extruding the outer cover.

9. The method of claim 3, wherein applying adhesive caps includes dipping both the distal and proximal ends of the lead into a liquid thermoplastic polyurethane elastomer adhesive.

10. The method of claim 9, wherein applying an adhesive caps includes initially dipping the distal or proximal ends of the lead into liquid thermoplastic polyurethane elastomer adhesive having a first percent solids content of polyurethane and then subsequently dipping the distal or proximal ends of the lead into liquid thermoplastic polyurethane elastomer adhesive having a second percent solids content of polyurethane, wherein the second solids content percent is larger than the first.

11. The method of claim 9, wherein applying an adhesive caps includes initially dipping the distal or proximal ends of the lead into liquid thermoplastic polyurethane elastomer adhesive and then subsequently applying a mold release agent and placing the distal end into a mold cavity to shape the end.

12. The method of claim 9, wherein applying an adhesive caps includes initially dipping the distal or proximal ends of the lead into liquid thermoplastic polyurethane elastomer adhesive, the adhesive comprising a polyurethane solid dissolved in a solvent, the solids content having a percent range from 2 percent to 30 percent.

13. The method of claim 9, wherein the liquid thermoplastic polyurethane elastomer adhesive includes a solvent such that the adhesive penetrates up into the plurality of conductors under capillary action.

14. A lead body comprising:
a plurality of individual conductors between a proximal end and a distal end of the lead body;
a plurality of electrodes, wherein each one electrode of the plurality of electrodes is coupled to each one of the plurality of individual conductors;
an outer cover over the plurality of individual conductors and over the electrodes in a substantially cylindrical shape with an outer diameter, the outer cover having a distal opening in the cover at the distal end, the distal opening having a same diameter as the outer cover;
wherein the outer cover comprises a first thermoplastic material that hermetically seals the lead body; and
an adhesive cap coupled to the outer cover and fully over the distal opening thereby closing the distal end, wherein the adhesive comprises the first thermoplastic material.

15. The lead body of claim 14, wherein the outer cover further comprises a proximal opening in the outer cover at the proximal end, the proximal opening having a same diameter as the outer cover.

16. The lead body of claim 15 further comprising an adhesive cap coupled to the outer cover and fully over the proximal opening thereby closing the proximal end, wherein the adhesive comprises the first thermoplastic material.

17. The lead body of claim 16, wherein the adhesive cap coupled the outer cover and over the proximal and distal openings has the same diameter as the diameter of both the proximal and distal openings.

18. The lead body of claim 14 further comprising a plurality of proximal rings, wherein each ring of the plurality of proximal rings is coupled to one of the plurality of individual conductors.

19. The lead body of claim 14, wherein the first thermoplastic material is a medical grade thermoplastic polyurethane elastomer.
